(19) United States
(12) Patent Application Publication
(10) Pub. No.: US 2024/0390650 A1
(43) Pub. Date: Nov. 28, 2024

(54) APPARATUS AND METHOD FOR WRIST AND CATHETER STABILIZATION DURING RADIAL ACCESS

(71) Applicant: Board of Regents of the University of Nebraska, Lincoln, NE (US)

(72) Inventors: Carlos Alvarez, Omaha, NE (US); William Thorell, Omaha, NE (US); Daniel Surdell, Omaha, NE (US); Andrew Gard, Omaha, NE (US); Landon Ehlers, Omaha, NE (US)

(73) Assignee: Board of Regents of the University of Nebraska, Lincoln, NE (US)

(21) Appl. No.: 18/696,670

(22) PCT Filed: Oct. 14, 2022
(86) PCT No.: PCT/US2022/078100

§ 371 Date: Mar. 28, 2024

Related U.S. Application Data
(60) Provisional application No. 63/256,032, filed on Oct. 15, 2021.

(51) Int. Cl. A61M 25/02

(52) U.S. Cl. CPC A61M 25/02

(57) ABSTRACT

An apparatus for wrist and catheter stabilization during radial artery access includes a wrist support member and a catheter support member connected by an adjustable coupler. The wrist support member comprises a first platform with a curved surface configured to cradle a wrist in a supine or neutral position and an inclined portion to motivate extension or ulnar flexion of the wrist. The catheter support member comprises a second platform with a curved sidewall extending along at least one side of the second platform. The curved sidewall may be configured to direct a catheter toward a radial access point of the wrist when the catheter is extended across the platform portion of the catheter support member. The adjustable coupler is reconfigurable to control a height of the catheter support member from the wrist support member, as well as orientation and other height/distance parameters in some embodiments.

### CROSS-REFERENCE TO RELATED APPLICATIONS

The present application claims priority under 35 U.S.C. § 119 (e) to U.S. Provisional Application Ser. No. 63/256,032, filed Oct. 15, 2021, and titled “Height Adjustable Radial Platform,” which is incorporated herein by reference in its entirety.

### TECHNICAL FIELD

The present invention generally relates to medical devices, more specifically to a device that aids with wrist and catheter stabilization for radial access.

### BACKGROUND

Radial artery access for interventions and diagnostics has been shown to reduce complications when compared to the traditional femoral artery approach. For example, interventions accomplished via the radial artery carry a lower risk of bleeding complications, allow for sooner mobilization of patients post operation, have no requirements to hold pressure or use closure devices at the site of catheterization, and most importantly, increase patient comfort. Advantages of a transradial procedure have led to the acceptance of the use for cardiac and neurointerventions. However, such an approach is complex and requires several steps in order to traverse multiple vascular tortuosities to carry out the interventions or diagnostics.

To perform a transradial procedure, a practitioner uses the radial artery in the left or right wrist of a patient as the entry point for the catheter. The practitioner typically stands on the right side of the patient and therefore, uses the right radial artery. In certain cases, however, the procedure needs to be done from the left arm, or through the left radial artery. The practitioner threads a thin hollow catheter through the patient's network of arteries in the arm in a retrograde fashion. For neurointerventions, the catheter is then threaded into the neck eventually reaching the patient's brain. For cardiac interventions, the catheter is threaded into the patient's chest, eventually reaching the patient's heart. Accordingly, the patient needs to be prepared and positioned in a supine position. The patient's arm and wrist need to be immobilized. To achieve this, devices such as radial arm-boards have been proposed, and designed, to offer support for the patient's arm during the procedure. Most of these arm-supports are designed to only support radial access procedures in which the wrist is placed in a supine position. The patient's wrist can either be placed in a supine or neutral position, although the neutral position is preferred. Positioning the hand in a neutral relaxed handshake posture relieves tension on the radial artery and obviates forceful restrained positioning of the patient's hand, thus increasing comfort. Devices which position the wrist in supination increase the difficulty of radial access and can lead to wrist discomfort.

### SUMMARY

An apparatus for wrist and catheter stabilization during radial artery access is disclosed. In embodiments, the apparatus includes a wrist support member and a catheter support member connected by an adjustable coupler. The wrist support member comprises a first platform with a curved surface configured to cradle a wrist in a supine or neutral position and an inclined portion to motivate extension or ulnar flexion of the wrist. The catheter support member comprises a second platform with a curved sidewall extending along at least one side of the second platform. The curved sidewall may be configured to direct a catheter toward a radial access point of the wrist when the catheter is extended across the platform portion of the catheter support member. The adjustable coupler is reconfigurable to control a height of the catheter support member from the wrist support member, as well as orientation and other height/distance parameters in some embodiments.

A method for wrist and catheter stabilization during radial artery access is also disclosed. In implementations, the method includes, but is not limited to, steps of: placing a wrist upon a wrist support member comprising a first platform with a curved surface configured to cradle the wrist in a supine or neutral position and an inclined portion to motivate extension or ulnar flexion of the wrist; extending a catheter across a catheter support member comprising a second platform with a curved sidewall extending along at least one side of the second platform, the curved sidewall configured to direct the catheter toward a radial access point of the wrist; and reconfiguring an adjustable coupler connecting the wrist support member and the catheter support member to control one or more height, distance, or orientation parameters. It is noted that the foregoing steps may be performed in another order or in parallel with one another. For example, the reconfiguration of the adjustable coupler may precede or overlap with the wrist placement step.

This Summary is provided solely as an introduction to subject matter that is fully described in the Detailed Description and Drawings. The Summary should not be considered to describe essential features nor be used to determine the scope of the Claims. Moreover, it is to be understood that both the foregoing Summary and the following Detailed Description are example and explanatory only and are not necessarily restrictive of the subject matter claimed.

### BRIEF DESCRIPTION OF THE DRAWINGS

The detailed description is provided with reference to the accompanying figures. The use of the same reference numbers in different instances in the description and the figures may indicate similar or identical items. Various embodiments or examples (“examples”) of the present disclosure are disclosed in the following detailed description and the accompanying drawings. The drawings are not necessarily to scale. In general, operations of disclosed processes may be performed in an arbitrary order, unless otherwise provided in the claims.

FIG. 1 is a left side perspective view of an apparatus for wrist and catheter stabilization during radial artery access, in accordance with one or more embodiments of this disclosure.

FIG. 2 is a right side perspective view of the apparatus for wrist and catheter stabilization during radial artery access, in accordance with one or more embodiments of this disclosure.

FIG. 3 is a left side elevation view of the apparatus for wrist and catheter stabilization during radial artery access, in accordance with one or more embodiments of this disclosure.

FIG. 4 is a right side perspective view of the apparatus for wrist and catheter stabilization during radial artery access, in accordance with one or more embodiments of this disclosure.

FIG. 5 is a proximal end view of the apparatus for wrist and catheter stabilization during radial artery access, in accordance with one or more embodiments of this disclosure.

FIG. 6 is a distal end view of the apparatus for wrist and catheter stabilization during radial artery access, in accordance with one or more embodiments of this disclosure.

FIG. 7 is a bottom view of the apparatus for wrist and catheter stabilization during radial artery access, in accordance with one or more embodiments of this disclosure.

FIG. 8 is a top view of the apparatus for wrist and catheter stabilization during radial artery access, in accordance with one or more embodiments of this disclosure.

FIG. 9 is an exploded view of the apparatus for wrist and catheter stabilization during radial artery access, in accordance with one or more embodiments of this disclosure.

FIG. 10 is an environmental view of the apparatus for wrist and catheter stabilization during radial artery access coupled to a bed rail, in accordance with one or more embodiments of this disclosure.

FIG. 11 is an environmental view of the apparatus for wrist and catheter stabilization during radial artery access coupled to and/or placed upon or above an arm board, in accordance with one or more embodiments of this disclosure.

FIG. 12 is an environmental view of the apparatus for wrist and catheter stabilization during radial artery access in an example use scenario, in accordance with one or more embodiments of this disclosure.

### DETAILED DESCRIPTION

The present disclosure describes an apparatus and method for stabilizing a wrist and a catheter during radial access interventional procedures and/or diagnostics. The apparatus is designed to support both right-sided and left sided interventions to place the wrist preferably in a neutral positioning with gentle ulnar flexion of the wrist, alternatively in supine positioning with gentle extension of the wrist, or alternatively in an intermediate positioning with gentle ulnar flexion and/or extension of the wrist. Current radial access setup typically requires a long board down the side of the patient, which decreases easy access to controls, and often requires a second operator or technician. Catheters need to be stabilized with towels and clips to prevent falling to the side. The presently disclosed apparatus is designed to prevent catheter falling and instability without the use of catheter clips and towels down the side of the bed and to comfortably position a patient's wrist for radial access interventions.

The apparatus is configured to facilitate the securement of the patient's wrist and catheter for a radial procedure as well as to accommodate for the re-positioning of the patient's wrist. The apparatus may generally comprise a wrist support member/platform defining a surface which is sized to support the wrist of the patient with a custom curvature for gentle ulnar flexion or extension of the wrist, a catheter support member/platform to direct a catheter toward a radial access point of the wrist, and an adjustable coupler that connects the wrist and catheter support members/platforms and is reconfigurable to adjust one or more height, distance, and/or orientation (e.g., tilt) parameters of the apparatus. The height adjustable catheter support member/platform can unobtrusively and gently guide catheters toward the radial access point of the wrist on an imaging table/bed, decreasing time needed for clipping and weighing down devices. This eliminates need for cumbersome boom arms and clamps and is easily adaptable to the situation.

Various embodiments and implementations of the apparatus are described below with reference to the accompanying drawings.

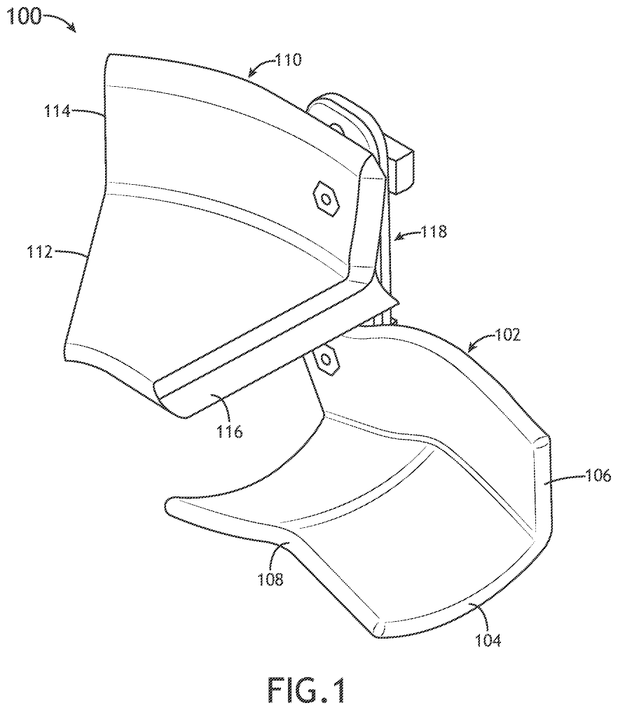

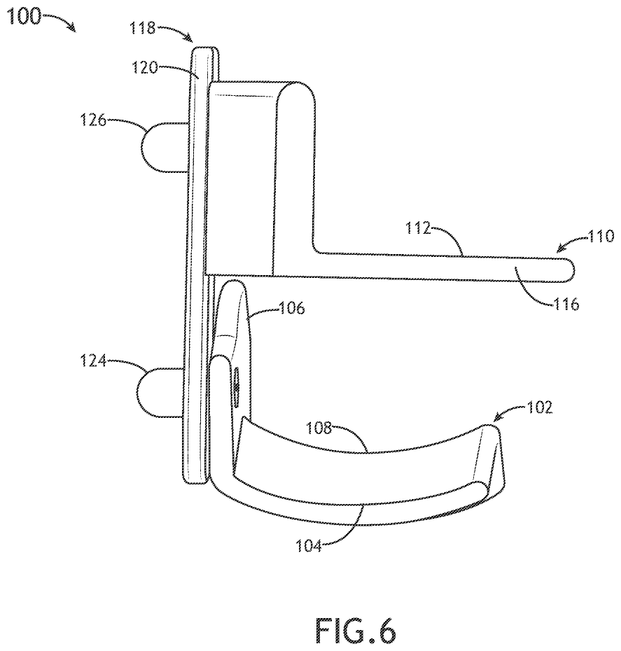

FIGS. 1 through 12 illustrate an apparatus 100 for wrist and catheter stabilization during radial artery access, in accordance with one or more embodiments of the present disclosure. The apparatus 100 may be used for a number of interventional procedures/diagnostics. For example, the apparatus 100 may be used for wrist and catheter stabilization in neurointerventional procedures including but not limited to, treatment of strokes, brain aneurysms and abnormal blood vessels, cerebrovascular disease, carotid stenosis, seizures, benign and malignant tumors, tinnitus, and for neurointerventional diagnostics. For further example, the apparatus 100 may be used for wrist and catheter stabilization in cardiac interventional procedures including but not limited to, angioplasty, stent placement, catheter ablation, heart valve surgery, bypass surgery, and for cardiac interventional diagnostics.

In some embodiments, the apparatus 100 is made of disposable (preferably biodegradable) plastic for single use, or any other single-use material or combination of materials. In other embodiments, the apparatus 100 may be composed of a reusable material, such as sterilizable carbon fiber nylon, carbon fiber alloy, radiolucent carbon fiber alloy, titanium, titanium alloy, or any other sterilizable metal, plastic, and/or composite material, or combination of materials.

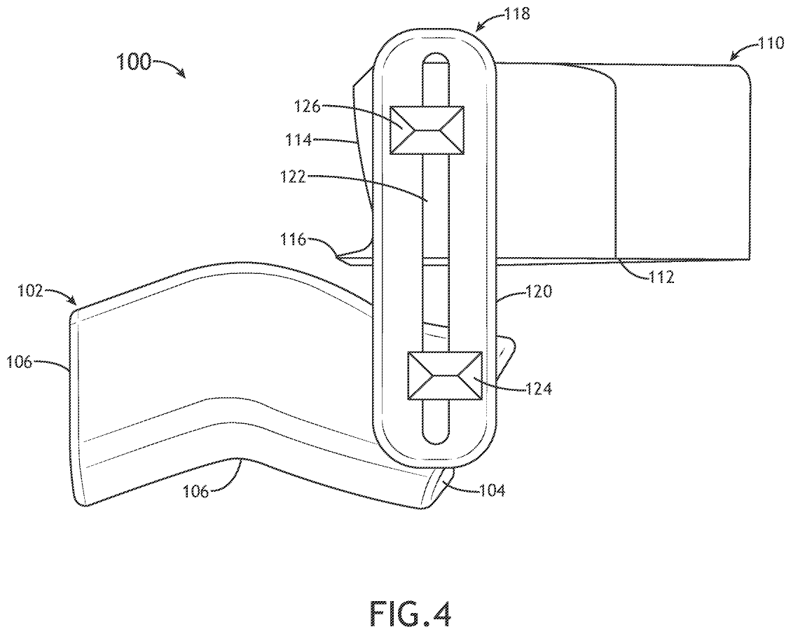

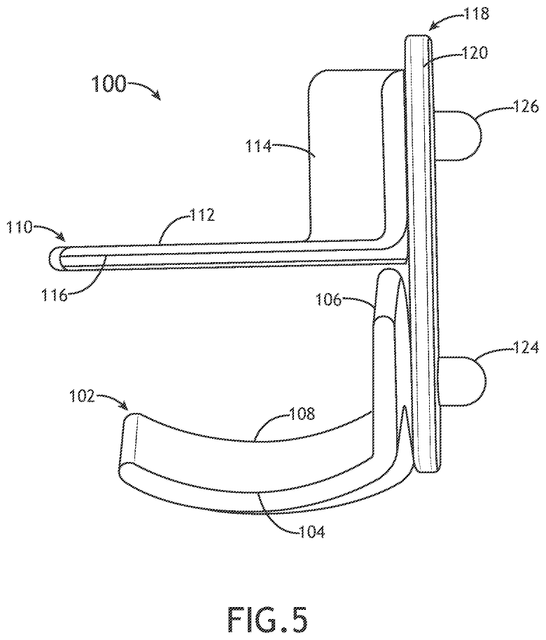

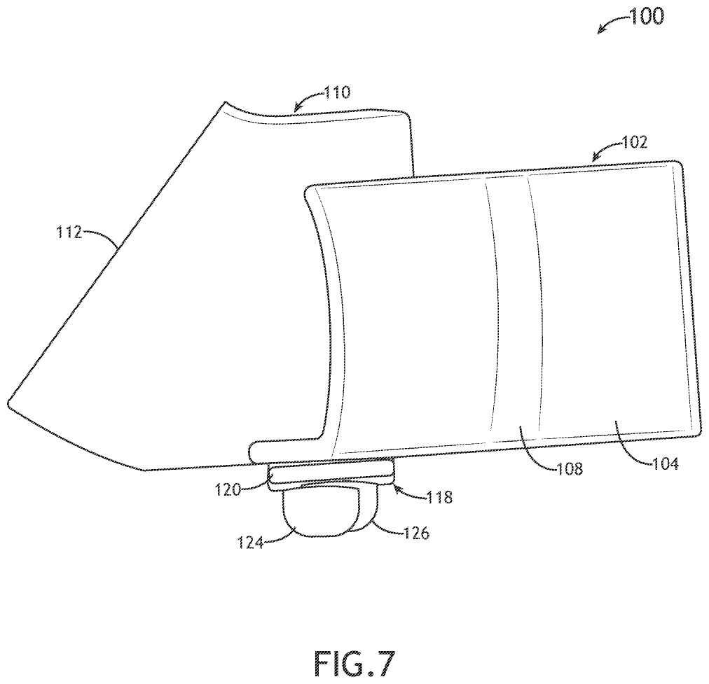

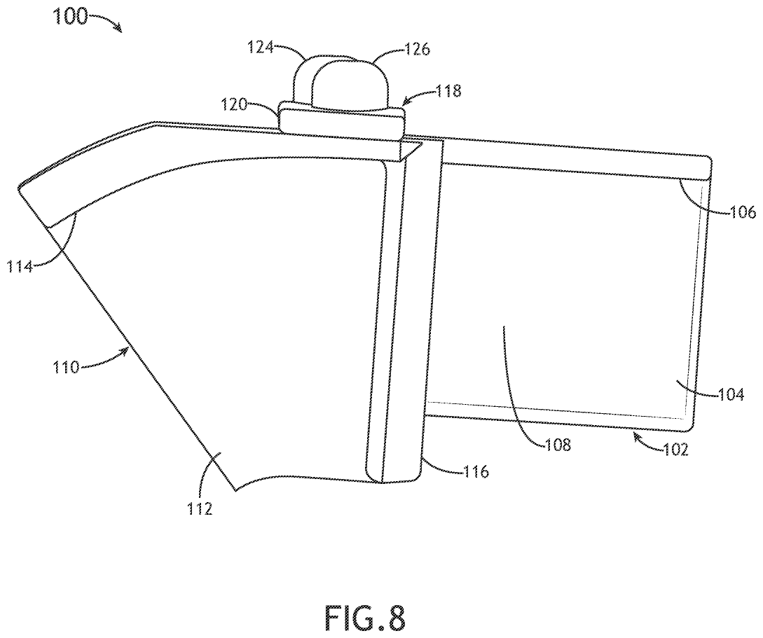

As shown in FIG. 1, the apparatus 100 includes a wrist support member 102 and a catheter support member 110 connected by an adjustable coupler 118. In embodiments, the wrist support member 102 comprises a first platform 104 with a curved surface configured to cradle a wrist in a supine or neutral position. For example, as shown in FIGS. 4 and 5, the surface of the platform 104 may be at least partially, all, or mostly concave. As shown in FIGS. 7 and 8, the perimeter of the platform may be substantially rectangular; although other shapes/geometries may be appropriate.

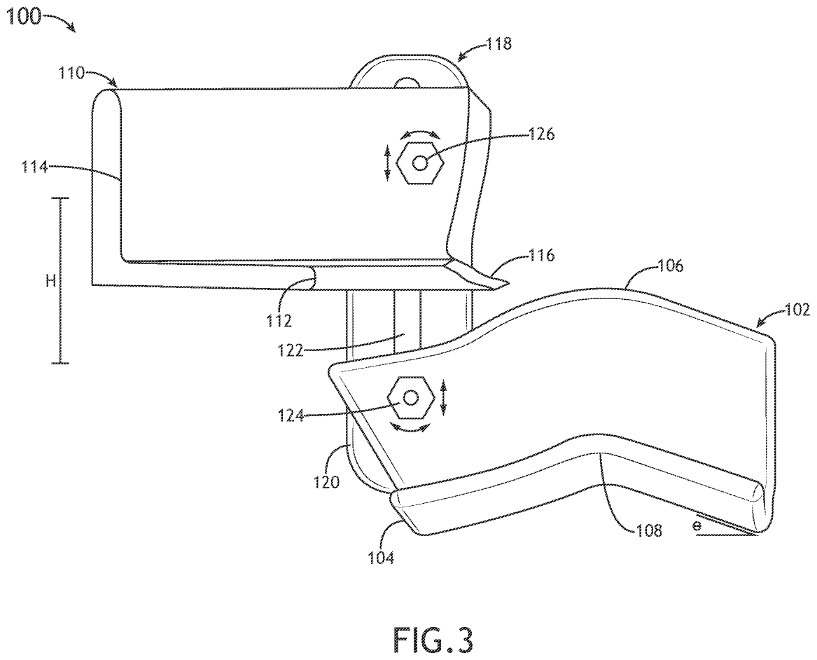

Referring now to FIG. 3, the platform 104 further includes an inclined portion 108 configured to motivate extension or ulnar flexion of the wrist. For example, the inclined portion 108 may comprise a ramp or bump in (or on) the surface of the platform 104. The inclined portion 108 may rise at an angle θ from the proximal end of the platform 104. In some embodiments, the angle θ is between 5 and 90 degrees, preferably between 15 and 60 degrees. The foregoing dimensions and ranges are provided as examples and are not intended as limitations on the present invention unless otherwise stated in the claims.

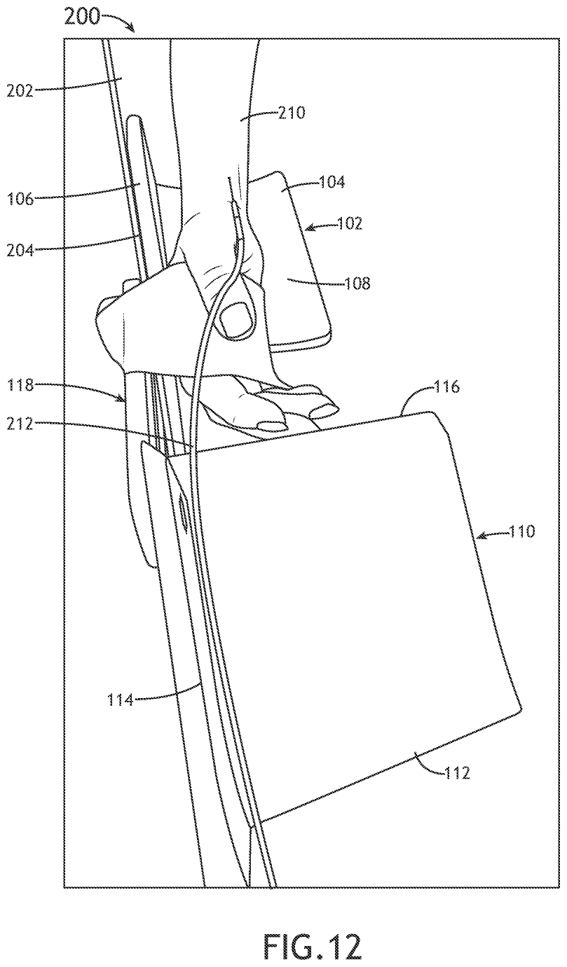

The wrist support member 102 may further include a sidewall 106 extending along at least one side of the platform 104. For example, the sidewall 106 may extend upwardly from the platform 104 as part of a common printed/molded structure or may be separately manufactured and attached to the platform 104. In some embodiments, the sidewall 106 extends at least partially, entirely, or mostly along the right or left side of the platform 104. The sidewall 106 may also be curved. As shown in FIG. 12, this curvature can assist in positioning a wrist 210 in a neutral position. In addition to aiding in the proper positioning of the wrist 210, the sidewall 106 may also help prevent the wrist 210 from rolling off of the platform 104.

In preferred embodiments, the length of the platform 104 may generally be between 10 cm and 30 cm, preferably between 15 cm and 25 cm. The width of the platform 104 may be between 5 cm and 20 cm, preferably between 10 cm and 15 cm. The thickness/height of the platform 104 may be between 2 cm and 12 cm, preferably between 3 cm and 9 cm. In a preferred example embodiment, the length of the platform 104 is about 20 cm, the width of the platform 104 is about 12 cm, and the thickness/height of the platform 104 is about 4 cm. The foregoing dimensions and ranges are provided as examples and are not intended as limitations on the present invention unless otherwise stated in the claims.

Referring again to FIG. 1, the catheter support member 110 may comprise a second platform 112. In some embodiments, the platform 112 is substantially planar but can also be curved or tapered in other embodiments. As shown in FIGS. 7 and 8, the perimeter of the platform may be substantially wedge shaped; although other shapes/geometries may be appropriate.

Referring again to FIG. 1, the catheter support member 110 may further include a sidewall 114 extending along at least one side of the platform 112. For example, the sidewall 114 may extend upwardly from the platform 112 as part of a common printed/molded structure or may be separately manufactured and attached to the platform 112. In some embodiments, the sidewall 114 extends at least partially, entirely, or mostly along the right or left side of the platform 112. The sidewall 114 may also be curved. As shown in FIG. 12, this curvature can assist in directing a catheter 212 toward a radial access point of the wrist 210 when the catheter 212 is extended across the platform 112 of the catheter support member 110. In addition to aiding in the proper positioning of the catheter 212, the sidewall 114 may also help prevent the catheter 212 from rolling off of the platform 112. In some embodiments, the platform 112 may also have a tapered edge 116 at a proximal end of the platform 112 to gently direct the catheter 212 downwardly towards the radial access point of the wrist 210.

In preferred embodiments, the length of the platform 112 may generally be between 5 cm and 35 cm, preferably between 10 cm and 30 cm. The width of the platform 112 may be between 5 cm and 20 cm, preferably between 9 cm and 16 cm. The thickness/height of the platform 112 may be between 2 cm and 12 cm, preferably between 4 cm and 9 cm. In a preferred example embodiment, the length of the platform 112 is about 15 cm, the width of the platform 112 is about 14 cm, and the thickness/height of the platform 112 is about 8 cm. In another preferred example embodiment, the length of the platform 112 is about 23 cm, the width of the platform 112 is about 14 cm, and the thickness/height of the platform 112 is about 8 cm. The foregoing dimensions and ranges are provided as examples and are not intended as limitations on the present invention unless otherwise stated in the claims.

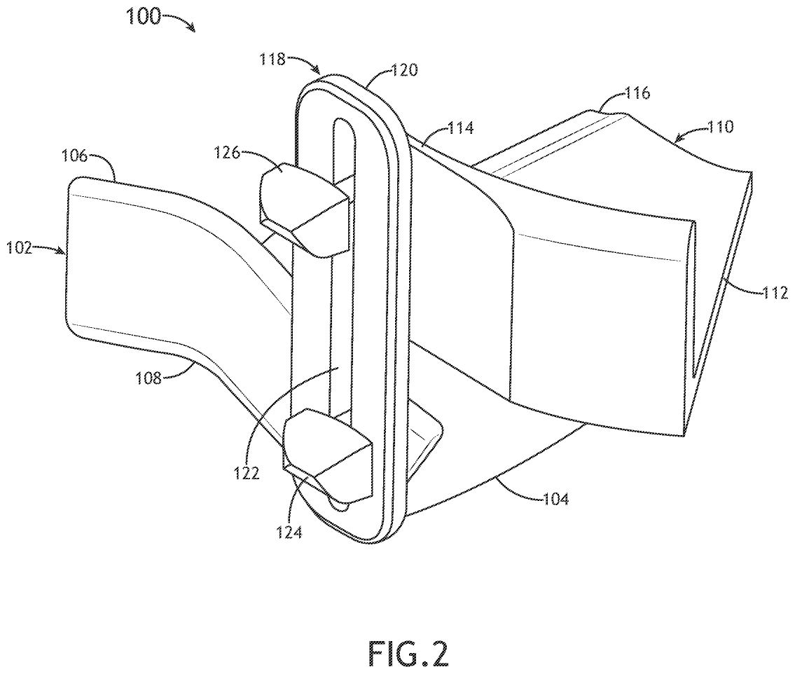

The wrist support member 102 and the catheter support member 110 are connected by the adjustable coupler 118, which is reconfigurable to control a height of the catheter support member 110 from the wrist support member 102, as well as orientation and other height/distance parameters in some embodiments. As shown in FIG. 2, the adjustable coupler 118 may include an elongate body 120 with an elongate slot 122 spanning at least a portion (preferably most) of the elongate body 120. The adjustable coupler 118 further includes a first fastener 124 configured to connect the wrist support member 102 to the adjustable coupler 118 and a second fastener 126 configured to connect the catheter support member 110 to the adjustable coupler 118. For example, the fasteners 124 and 126 may include screws/bolts configured to extend through the elongate slot 122 and through respective holes in the wrist support member 102 and the catheter support member 110 so that the wrist support member 102 and the catheter support member 110 are slidably coupled to elongate body 120 via the elongate slot 122. In some embodiments, the elongate slot 122 may be replaced by multiple elongate slots, for example, separate elongate slots for each of the support members 102 and 110.

In preferred embodiments, the length of the adjustable coupler 118 (i.e., the length of the elongate body 120) may be between 10 cm and 40 cm, preferably between 15 cm and 35 cm. The width of the adjustable coupler 118 (i.e., the width of the elongate body 120) may be between 2 cm and 8 cm, preferably between 4 cm and 6 cm. In a preferred example embodiment, the length of the adjustable coupler 118 is about 18 cm, and the width of the adjustable coupler 118 is about 5 cm. In another preferred example embodiment, the length of the adjustable coupler 118 is about 30 cm, and the width of the adjustable coupler 118 is about 5 cm. The foregoing dimensions and ranges are provided as examples and are not intended as limitations on the present invention unless otherwise stated in the claims.

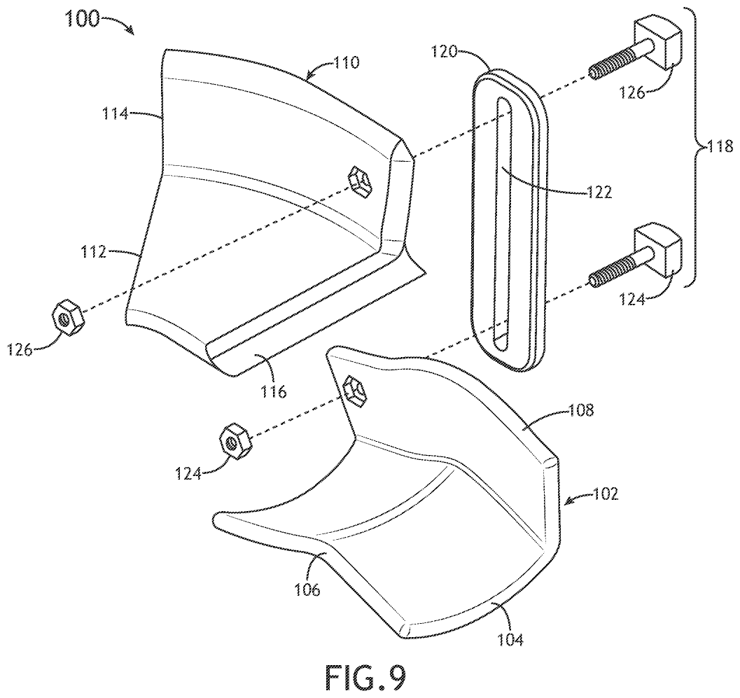

As shown in FIG. 9, the fasteners 124 and 126 may be configured to extend through holes formed in the sidewalls 108 and 114 of the wrist support member 102 and the catheter support member 110, respectively. Alternatively, fastener 124 may be configured to extend through a respective hole formed in platform 104, and/or fastener 126 may be configured to extend through a respective hole formed in platform 112. In some embodiments, the fasteners 124 and 126 are cooperatively threaded nuts and bolts as shown in FIG. 9. In preferred embodiments, the nuts (e.g., hex nuts) are at least partially or fully recessed within the respective sidewalls 106 and 114 of the wrist support member 102 and the catheter support member 110. This configuration allows for increased torque and enhanced stability of the respective platforms 104 and 112 of the wrist support member 102 and the catheter support member 110. Alternatively, the nuts may not be recessed, which can also have certain advantages such as less shape complexity and hence easier manufacturing of the wrist support member 102 and the catheter support member 110. In other embodiments, the fasteners 124 and 126 may comprise screws, clips, ratcheting fasteners, snap or pressure fit connectors, or any other type of fastener that can be selectively unfixed (e.g., untightened or unsecured) and refixed (e.g., re-tightened or re-secured) to make adjustments.

Referring now to FIGS. 3 and 4, the adjustable coupler 118 is reconfigurable to control one or more height, distance, and/or orientation (e.g., tilt) parameters of the apparatus 100. In some embodiments, the wrist support member 102 and the catheter support member 110 can each be adjusted to control their respective height and tilt, as well as (horizontal) distance from one another. Alternatively, the adjustable coupler 118 may only accommodate a subset of these controls. In preferred embodiments, the adjustable coupler 118 has two principal features that enable a wide array of adjustments: (1) the fasteners 124 and 126 can slide up and down the elongate body 120 (e.g., within the elongate slot 122) of the adjustable coupler 118; and (2) the wrist support member 102 and the catheter support member 110 can be tilted about respective pivots (e.g., the respective fasteners 124 and 126) that secure the support members to the adjustable coupler 118. In some embodiments, the adjustable coupler 118 may have limited functionality because the wrist support member 102, the catheter support member 110, and/or their respective fasteners 124 and 126 lack one or some of the aforementioned features and are nonadjustable or only partially adjustable as a result.

Various examples of height, distance, and/or orientation adjustments are described below.

In embodiments, the adjustable coupler 118 is reconfigurable to control a height H of the catheter support member 110 from the wrist support member 102. For example, the height H of the catheter support member 110 from the wrist support member 102 may be adjusted by unfixing the fastener 126 that connects the catheter support member 110 to the elongate body 120/elongate slot 122 of the adjustable coupler 118 and refixing the fastener 126 at a selected height from the wrist support member 102.

Additionally, the adjustable coupler 118 may be reconfigurable to control an orientation (e.g., tilt) of the catheter support member 110 relative to the wrist support member 102, or relative to the adjustable coupler 118 itself. For example, the orientation of the catheter support member 110 relative to the wrist support member 102 may be adjusted by tilting/pivoting the catheter support member 110 (e.g., tilting platform 112) about the fastener 126 to reach a selected tilt angle.

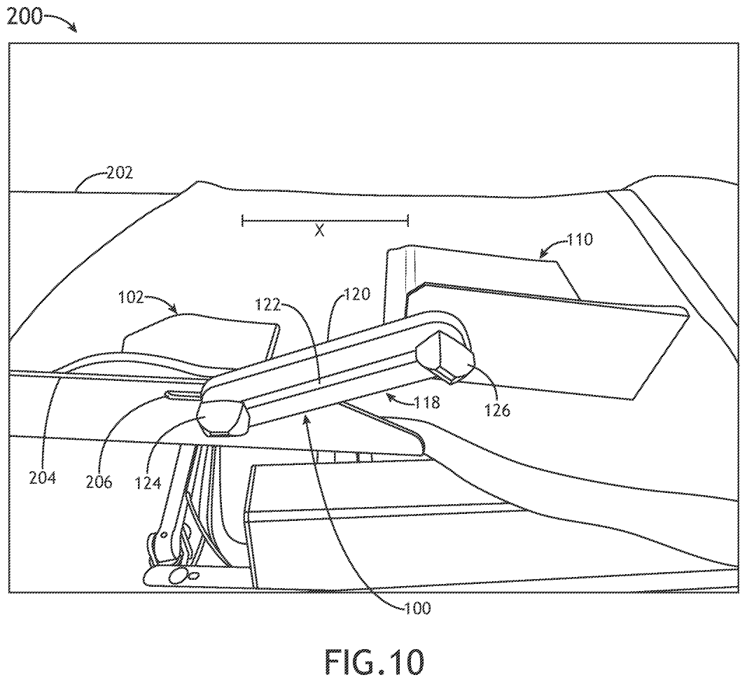

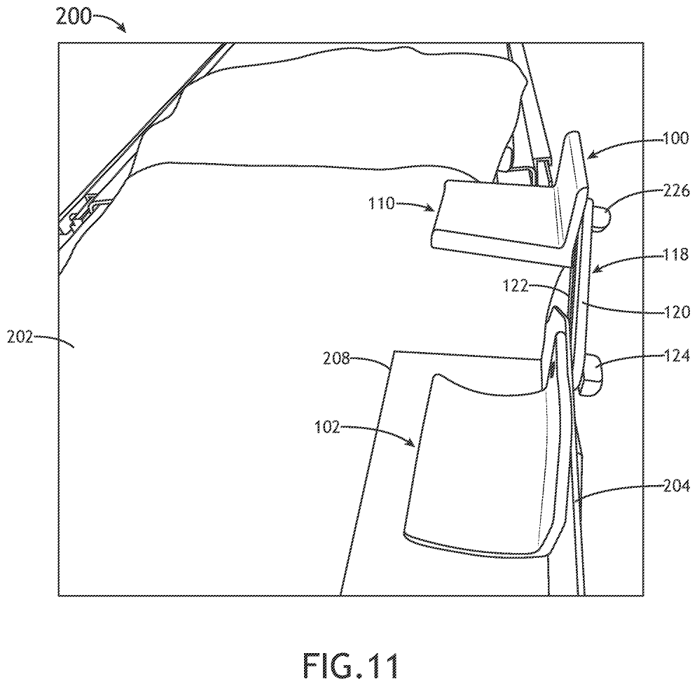

As shown in the example environments 200 illustrated in FIGS. 10 and 11, the adjustable coupler 118 may be further configured to connect to bed rail 204 of a bed 202 and/or an arm board 208. For example, the adjustable coupler 118 may be configured to connect the apparatus 100 to the bed rail 204 (e.g., via a slot 206 or hole formed in the bed rail 204), arm board 208 (e.g., via a slot or hole formed in the arm board 204), or any other anchoring mechanism using the same fastener 124 that connects the wrist support member 102 to the adjustable coupler 118. Alternatively, the apparatus 100 may be placed on the bed 202 (or procedure table) or arm board 208 without being secured to an anchoring mechanism via fastener 124. In such cases, the platform 104 of the wrist support member 102 acts to support the apparatus 100.

In embodiments where the adjustable coupler 118 is secured to an anchoring mechanism (e.g., via fastener 124), the adjustable coupler 118 may be reconfigurable to control an orientation of the wrist support member 102 relative to the bed 202, the arm board 208, and/or the bed rail 204. For example, the orientation (e.g., tilt) of the wrist support member 102 relative to the bed 202, arm board 208, and/or bed rail 204 may be adjusted by tilting/pivoting the wrist support member 102 (e.g., tilting platform 104) about the fastener 124 to reach a selected tilt angle.

In either configuration (anchored or non-anchored), the adjustable coupler 118 may be reconfigurable to control a horizontal distance between the wrist support member 102 and the catheter support member 110. For example (see FIG. 10), the horizontal distance X between the wrist support member 102 and the catheter support member 110 can be adjusted by tilting (e.g., slanting or leaning forward) the adjustable coupler 118 (i.e., tilting the elongate body 120) to increase the horizontal distance X while the wrist support member 102 and the catheter support member 110 are tilted/pivoted about their respective fasteners 124 and 126 without changing relative orientation to one another. Conversely, the adjustable coupler 118 can be brought into vertical alignment (e.g., as shown in FIG. 4) or raised toward vertical alignment to decrease the horizontal distance between the wrist support member 102 and the catheter support member 110.

FIG. 10 illustrates an example environment 200 including a procedure table/bed 202 with a rail 204 including a slot 206 to which the apparatus 100 is coupled via the adjustable coupler 118. FIG. 11 is another illustration of the example environment 200 now including an arm board 208 to which the apparatus 100 is coupled via the adjustable coupler 118. In general, the apparatus 100 can be used with a number of catheter-based systems including but not limited to, arm boards, radiation scatter protective shields, and compression devices. For example, the adjustable coupler 118 may be configured to connect (via fastener 124) to standard arm boards that are included in the Siemens or Phillips biplane angiography setup through the restraint strap slots.

FIG. 12 illustrates the example environment 200 in an example use scenario where a patient's wrist 210 is placed on the wrist support member 102 (i.e., on platform 104), and a catheter 212 is extended across the catheter support member 110 (i.e., across platform 114). In some embodiments, the apparatus 100 further includes one or more straps or belts configured to secure the wrist 210 to the platform 104 and/or against the sidewall 106 of the wrist support member 102. The wrist support member 102 can also optionally include a soft pad or cushion disposed upon the platform 104 to provide a more comfortable interface for the wrist 210. In some embodiments, the apparatus 100 further includes one or more straps, belts, straps, catheter clips, or catheter hooks configured to secure the catheter 212 to the platform 112 and/or against the sidewall 114 of the catheter support member 110.

A method of using the apparatus may include, but is not limited to, steps of: placing the apparatus 100 on and/or connecting the apparatus 100 to the procedure table/bed 202; placing the patient's wrist upon the platform 104 of the wrist support member 102 in a supine or neutral position; extending the catheter 212 across the platform 112 of the catheter support member 110 so that the curved sidewall 114 directs the catheter 212 toward a radial access point of the patient's wrist 210; and reconfiguring the adjustable coupler 118 to control one or more height, distance, or orientation parameters of the wrist support member 102 and the catheter support member 110, which may be independently adjusted to optimize wrist and catheter positions for a radial access procedure. It is noted that the foregoing steps may be performed in another order or in parallel with one another. For example, the reconfiguration of the adjustable coupler may precede or overlap with the wrist placement step.

Although the technology has been described with reference to the embodiments illustrated in the attached drawing figures, equivalents may be employed, and substitutions may be made herein without departing from the scope of the technology as recited in the claims. Components illustrated and described herein are examples of devices and components that may be used to implement the embodiments of the present invention and may be replaced with other devices and components without departing from the scope of the invention. Furthermore, any dimensions, degrees, and/or numerical ranges provided herein are to be understood as non-limiting examples unless otherwise specified in the claims.

What is claimed is:

1. An apparatus for wrist and catheter stabilization during radial artery access, comprising:
a wrist support member comprising a first platform with a curved surface configured to cradle a wrist in a supine or neutral position and an inclined portion to motivate extension or ulnar flexion of the wrist;
a catheter support member comprising a second platform with a curved sidewall extending along at least one side of the second platform, the curved sidewall configured to direct a catheter toward a radial access point of the wrist; and
an adjustable coupler connecting the wrist support member and the catheter support member, the adjustable coupler being reconfigurable to control a height of the catheter support member from the wrist support member.

2. The apparatus of claim 1, wherein the adjustable coupler is reconfigurable to control the height of the catheter support member from the wrist support member by unfixing a fastener that connects the catheter support member to an elongate slot in the adjustable coupler and refixing the fastener at a selected height from the wrist support member.

3. The apparatus of claim 1, wherein the adjustable coupler is also reconfigurable to control an orientation of the catheter support member relative to the wrist support member.

4. The apparatus of the claim 3, wherein the adjustable coupler is reconfigurable to control the orientation of the catheter support member relative to the wrist support member by tilting the catheter support member about a fastener that connects the catheter support member to an elongate slot in the adjustable coupler to reach a selected tilt angle.

5. The apparatus of claim 1, wherein the adjustable coupler also connects to at least one of an arm board or a bed rail.

6. The apparatus of claim 5, wherein the adjustable coupler is also reconfigurable to control an orientation of the wrist support member relative to the at least one of the arm board or the bed rail.

7. The apparatus of the claim 6, wherein the adjustable coupler is reconfigurable to control the orientation of the wrist support member relative to the at least one of the arm board or the bed rail by tilting the wrist support member about a fastener that connects the wrist support member to an elongate slot in the adjustable coupler to reach a selected tilt angle.

8. The apparatus of claim 1, wherein the adjustable coupler is also reconfigurable to control a horizontal distance between the catheter support member and the wrist support member.

9. The apparatus of the claim 8, wherein the adjustable coupler is reconfigurable to control the horizontal distance between the catheter support member and the wrist support member by tilting the adjustable coupler to increase the horizontal distance between the catheter support member and the wrist support member while the catheter support member and the wrist support members pivot about respective fasteners without changing relative orientation to one another.

10. The apparatus of claim 1, wherein the wrist support member further comprises a sidewall extending along at least one side of the first platform to assist with at least one of positioning or stabilizing the wrist.

11. The apparatus of claim 1, wherein the wrist support member further includes a soft pad disposed upon the first platform.

12. The apparatus of claim 1, further comprising:
one or more straps or belts configured to secure the wrist to the wrist support member.

13. The apparatus of claim 1, further comprising:
one or more straps, belts, catheter clips, or catheter hooks configured to secure the catheter to the catheter support member.

14. The apparatus of claim 1, wherein the wrist support member, the catheter support member, and the adjustable coupler are manufactured using plastic for single use.

15. The apparatus of claim 1, wherein the wrist support member, the catheter support member, and the adjustable coupler are manufactured using carbon fiber nylon, carbon fiber alloy, titanium, or titanium alloy for repeated use.

16. A method for wrist and catheter stabilization during radial artery access, comprising:
placing a wrist upon a wrist support member comprising a first platform with a curved surface configured to cradle the wrist in a supine or neutral position and an inclined portion to motivate extension or ulnar flexion of the wrist;
extending a catheter across a catheter support member comprising a second platform with a curved sidewall extending along at least one side of the second platform, the curved sidewall configured to direct the catheter toward a radial access point of the wrist; and
reconfiguring an adjustable coupler connecting the wrist support member and the catheter support member to control one or more height, distance, or orientation parameters.

17. The method of claim 16, wherein the adjustable coupler is reconfigured to control the height of the catheter support member from the wrist support member by unfixing a fastener that connects the catheter support member to an elongate slot in the adjustable coupler and refixing the fastener at a selected height from the catheter support member.

18. The method of the claim 16, wherein the adjustable coupler is reconfigured to control an orientation of the catheter support member relative to the wrist support member by tilting the catheter support member about a fastener that connects the catheter support member to an elongate slot in the adjustable coupler to reach a selected tilt angle.

19. The method of the claim 16, wherein the adjustable coupler is reconfigured to control an orientation of the wrist support member relative to at least one of an arm board or a bed rail by tilting the wrist support member about a fastener that connects the wrist support member to an elongate slot in the adjustable coupler to reach a selected tilt angle.

20. The method of the claim 16, wherein the adjustable coupler is reconfigured to control a horizontal distance between the catheter support member and the wrist support member by tilting the adjustable coupler to increase the horizontal distance between the catheter support member and the wrist support member while the catheter support member and the wrist support members pivot about respective fasteners without changing relative orientation to one another.
